(19) United States
(12) Patent Application Publication
(10) Pub. No.: US 2018/0318238 A1
(43) Pub. Date: Nov. 8, 2018

(54) METHODS FOR REDUCING SEQUELAE OF INTRA-DIALYTIC HYPOTENSION

(71) Applicants: Roy FARFARA, Nicosia (CY); Dobroslav MELAMED, Rishon Lesion (IL); Shlomo SADOUN, Rishon-Le-Zion (IL)

(72) Inventors: Roy FARFARA, Nicosia (CY); Dobroslav MELAMED, Rishon Lesion (IL); Shlomo SADOUN, Rishon-Le-Zion (IL)

(21) Appl. No.: 15/772,276

(22) PCT Filed: Oct. 30, 2016
(86) PCT No.: PCT/IL2016/051172

§ 371 Date: Apr. 30, 2018

Related U.S. Application Data
(60) Provisional application No. 62/249,281, filed on Nov. 1, 2015.

(51) Int. Cl. A61K 31/155; A61P 9/02

(52) U.S. Cl. CPC A61K 31/155; A61K 9/0019; A61P 9/02

(57) ABSTRACT

Provided are methods for using S-alkylisothiouronium derivatives, including S-ethylisothiouronium diethylphosphate, to alleviate, prevent and/or reduce a sequela of intra-dialytic hypotension.

### FIELD OF THE INVENTION

The present invention relates to the use of S-alkylisothiouronium derivatives for alleviating thrombotic events in patients in need thereof. In particular, the present invention relates to methods for reducing sequelae of intra-dialytic hypotension including vascular access thrombosis and drug induced hypertension.

### BACKGROUND OF THE INVENTION

Renal failure, also known as kidney failure or renal insufficiency, is a medical condition in which the kidneys fail adequately to filter waste products from the blood. The two main forms are acute kidney injury, which is often reversible with adequate treatment, and chronic kidney disease, which most often is irreversible. The last stage of chronic kidney disease, also known as end-stage renal disease (ESRD) ESRD require dialysis or kidney transplantation.

Dialysis is a life-extending procedure during which the patient's blood is cleansed by exchanging fluid and electrolytes across a membrane. Typically, 3-4 hour treatments (sometimes up to 5 hours for larger patients) are given 3 times a week. Twice-a-week sessions are limited to patients who have a substantial residual kidney function. Four sessions per week are often prescribed for larger patients, as well as patients who have trouble with fluid overload. During the dialysis, the patient is attached to a dialysis machine through vascular access points. The access points are surgically created veins used to remove and return blood during hemodialysis. Two types of vascular access, designed for long-term use, include the arteriovenous (AV) fistula and the AV graft. A third type of vascular access, the venous catheter, is for short-term use.

A fistula is a direct connection of an artery to a vein, surgically created from the patient's own blood vessels. The fistula causes extra pressure and extra blood to flow into the vein, making it grow large and strong, thereby providing easy, reliable access to blood vessels.

It has been shown that intradialytic hypotension is associated with arteriovenous fistula thrombosis in patients on hemodialysis (Chang, T. et al., J Am Soc Nephrol 22: 1526-1533, 2011).

Vascular access complications mainly consisting of thrombotic events are responsible for 17-25% of all hospitalizations in dialysis patients. Clotting of the access point can lead to their closure. As a result, patients are often necessitated to go through multiple access point, and when a patient runs out of access points, it becomes an emergency situation.

WO 98/13036 to Mizrakh et al. discloses the use of S-alkylisothiouronium derivatives, as medicaments for increasing arterial blood pressure or for protecting subjects against hyperoxia. These compounds are suggested for the treatment of acute hypotension, e.g., shock conditions and chronic arterial hypotension or oxygen poisoning. The invention is exemplified by the hypertensive effect of S-ethylisothiouronium diethylphosphate (difetur) under various conditions.

WO 02/19961 to Barkan et al. discloses the use of S-alkylisothiouronium derivatives, for the prevention or treatment of headache, in particular migraine, as well as for the prevention of nausea and vomiting.

WO 2007/029255 to Barkan et al. relates to the use of S-alkylisothiouronium derivatives, including S-ethylisothiouronium diethylphosphate, for stabilizing blood pressure in hemodialysis patients. The compositions of the invention are disclosed as being effective in preventing hypotension in hemodialysis patients

However, the art fails to teach or suggest the use of S-alkylisothiouronium derivatives for reducing sequelae of intra-dialytic hypotension. Specifically, the art fails to suggest the use of S-alkylisothiouronium derivatives for preventing drug induced hypertension and for reducing incidents of vascular access thrombosis in hemodialysis patients and/or for preventing drug induced hypertension.

### SUMMARY OF THE INVENTION

The present invention provides methods and compositions for reducing sequelae of intra-dialytic hypotension. In particular, the present invention discloses for the first time that intra-dialytic administration of S-alkyl-isothiouronium derivatives reduce the incidence of vascular access thrombosis in hemodialysis patients. Advantageously, the present invention further discloses for the first time that intra-dialytic administration of S-alkyl-isothiouronium derivatives to human dialysis patients stabilizes the patient's blood pressure at normal blood pressure levels (normotension), while preventing drug induced hypertension.

This finding is surprising since WO 98/13036 demonstrated that intravenous administration of S-ethylisothiouronium diethylphosphate to dogs had a pronounced hypertensive effect at all doses used. WO 98/13036 further disclosed that the hypertensive reaction was significantly stronger and more prolonged in dogs with lower arterial blood pressure (hypotensive dogs) than in dogs with an initially higher arterial blood pressure level (hypertensive dogs).

The present invention further provides methods and compositions for extending the lifetime of vascular access points. Advantageously, this decreases the number of access points utilized per patient and thus reduces the risk of running out of access points.

Thus, according to one aspect, the present invention provides a method for reducing occurrence of vascular access thrombosis in a subject receiving hemodialysis comprising administering to the subject a therapeutically effective amount of a compound having the general formula I:

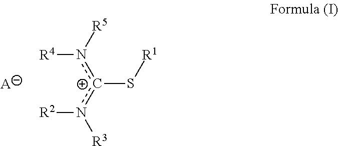

wherein,
R¹ is a linear or branched, saturated or unsaturated alkylene, comprising one to eight carbon atoms, optionally substituted with one or more substituents selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, an alkylene including linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
   and a pharmaceutically acceptable carrier or diluent.

According to some embodiments, reducing the incident of vascular access thrombosis comprises reducing the incidence of arteriovenous fistula thrombosis.

According to another aspect, the present invention provides a method for extending the lifetime of a vascular access point of a subject receiving hemodialysis comprising administering to the subject a therapeutically effective amount of a compound having the general formula I:

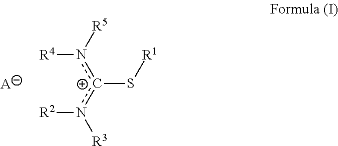

wherein,
R¹ is a linear or branched, saturated or unsaturated alkylene, comprising one to eight carbon atoms, optionally substituted with one or more substituents selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, an alkylene including linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
   and a pharmaceutically acceptable carrier or diluent.

According to some embodiments, the vascular access point comprises an arteriovenous fistula. According to some embodiments, extending the lifetime of a vascular access point comprises extending the lifetime of the access point by at least one week, by at least two weeks, by at least one month, by at least two months, or by at least six months, relative to an average lifetime of an untreated access point. Each possibility is a separate embodiment.

According to some embodiments, extending the lifetime of a vascular access point comprises extending the lifetime of the access point by at least one hemodialysis session, by at least two hemodialysis session, by at least three hemodialysis sessions, by at least five hemodialysis sessions, by at least 10 hemodialysis sessions, or by at least fifteen hemodialysis sessions relative to the average number of hemodialysis session per lifetime of an untreated vascular access point. Each possibility is a separate embodiment. As used herein, the term “hemodialysis session” refers to a single (typically out of three weekly) procedures of hemodialysis.

According to another aspect, the present invention provides a method for reducing a sequela of hypotension, the method comprising administering to the subject a therapeutically effective amount of a compound having the general formula I:

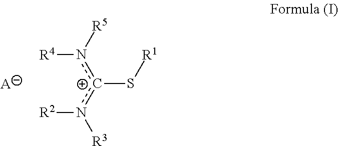

wherein,
R¹ is a linear or branched, saturated or unsaturated alkylene, comprising one to eight carbon atoms, optionally substituted with one or more substituents selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, an alkylene including linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
   and a pharmaceutically acceptable carrier or diluent.

According to some embodiments, the sequela of hypotension is selected from vascular access dysfunction, vascular access closure, vascular access thrombosis, drug induced hypertension and combinations thereof. Each possibility is a separate embodiment. According to some embodiments, vascular access dysfunction comprises arteriovenous fistula dysfunction. According to some embodiments, vascular access closure comprises arteriovenous fistula closure. According to some embodiments, vascular access thrombosis comprises arteriovenous fistula thrombosis. According to some embodiments, the subject is a hemodialysis patient.

According to one embodiment of the present invention, the physiologically acceptable anion is selected from the group consisting of an anion derived from a phosphorus containing acid, a phosphorus acid ester and a phosphorus acid amide, preferably the anion is derived from a mono or di-alkyl ester of a phosphate or phosphite.

In other embodiments the physiologically acceptable anion is selected from the group consisting of an anion derived from a phosphorus containing acid, a phosphorous acid ester, a phosphorous acid amide, acetate, adipate, alginate, citrate, aspartate, benzoate, benzenesulfonate, bitartarate, bisulfate, butyrate, camphorate, camphorsulfonate, digluconate, glycerophosphate, hemisulfate, heptanoate, hexanoate, fumarate, 2-hydroxyethanesulfonate, isothionate, lactate, maleate, methanesulfonate, nicotinate, 2-naphthalenesulfonate, oxalate, palmoate, pectinate, 3-phenylpropionate, pivalate, propionate, succinate, tartrate, thiocyanate, glutamate, bicarbonate, p-toluenesulfonate, chloride, bromide, iodide and undecanoate. Each possibility is a separate embodiment.

In yet other embodiments each of R², R³, R⁴ and R⁵ is hydrogen. In some embodiments R¹ is a linear or branched alkyl.

Accordingly, in one embodiment the S-alkylisothiouronium derivative is a compound of formula (II):

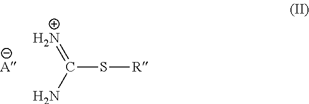

wherein
R″ is a straight or branched alkyl, optionally substituted by halogen; and
A″ (−) is an anion derived from a phosphorous containing acid.

The present invention further provides use of a compound having general formula (I) or (II) for the manufacture of a medicament for alleviating, preventing and/or for reducing the number of occurrences of vascular access thrombosis in hemodialysis patients.

According to some embodiments, the compound is selected from the group consisting of:

S-methylisothiouronium methylphosphite; S-methylisothiouronium dimethylphosphate; S-ethylisothiouronium metaphosphate; S-ethylisothiouronium ethylphosphite; S-ethylisothiouronium diethylphosphate; S-propylisothiouronium propylphosphite; S-isopropylisothiouronium metaphosphate; S-isopropylisothiouronium isopropylphosphite; S-butylisothiouronium dibutylphosphate; and S-isobutyl-isothiouronium isobutylphosphite. Each possibility is a separate embodiment.

In certain embodiments, the compound is S-ethylisothiouronium diethylphosphate.

According to still further features in the described preferred embodiments, the anti-thrombosis medicament is formulated for parenteral modes of administration. Among the parenteral routes of administration, particularly preferred formulations are suitable for injection, or infusion administration. Another preferred route of administration is oral administration.

According to one embodiment, the anti-thrombosis medicament is administered before the hemodialysis.

Additionally or alternatively, the anti-thrombosis medicament is administered during the hemodialysis. Additionally or alternatively, the anti-thrombosis medicament is administered after the hemodialysis.

According to some embodiments, the therapeutically effective amount suitable for injection, or infusion administration ranges between 0.1 and 5 mg/kg body weight. According to other embodiments, said therapeutically effective amount ranges between 0.1 and 2.4 mg/kg body weight. According to some embodiments, said therapeutically effective amount ranges between 0.3 and 2.4 mg/kg body weight. According to other embodiments, said therapeutically effective amount ranges between 0.5 and 1.8 mg/kg body weight. According to other embodiments, said therapeutically effective amount ranges between 0.5 and 1.2 mg/kg body weight.

According to other embodiments, the therapeutically effective amount suitable for oral administration ranges between 0.1 and 2.4 mg/kg body weight.

These and other embodiments of the present invention will become apparent in conjunction with the figures, description and claims that follow.

### BRIEF DESCRIPTION OF THE FIGURES

FIG. 1 show the effect of an injectable formulation of S-ethylisothiouronium diethylphosphate (TXB-104) on blood pressure during hemodialysis in baseline patients (grey dashed line) and in patients administered TXB-104 (black dashed line). The pharmacokinetics of TXB-104 is also shown (grey full line).

FIG. 2 schematically illustrates intra-dialytic blood pressure over time in patients treated with TXB-104 or with a conventional sympathomimetic drug.

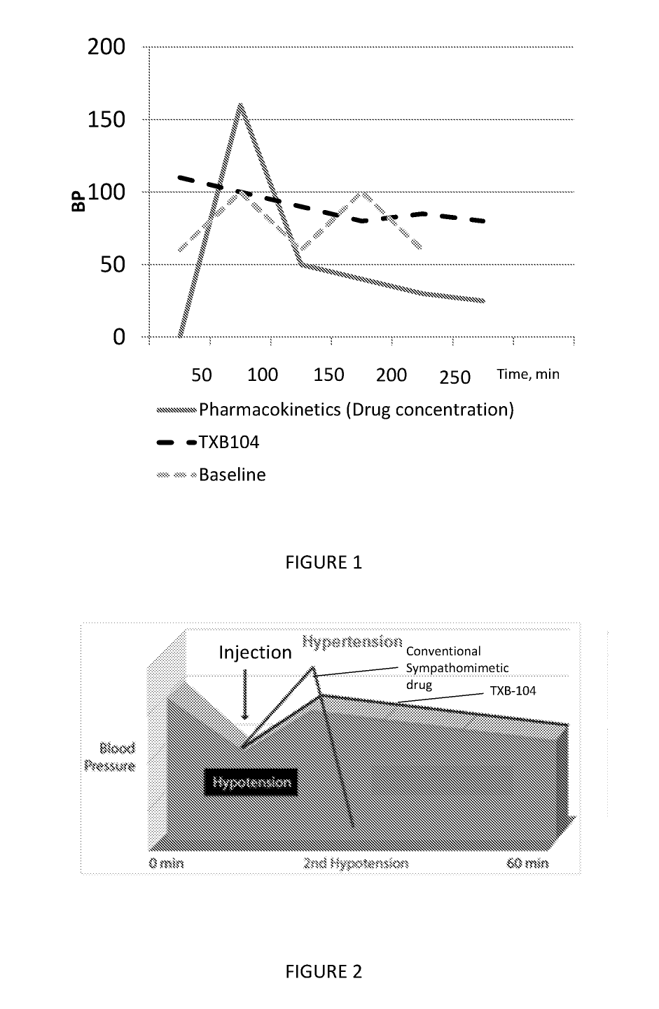

### DETAILED DESCRIPTION OF THE INVENTION

The present invention relates to the use of S-alkylisothiouronium derivatives, including, but not limited to, S-ethylisothiouronium diethylphosphate, also known by the trivial chemical name difetur, for alleviating, preventing and/or reducing the occurrence of vascular access thrombosis and/or for extending the lifetime of a vascular access point in hemodialysis patients.

The present invention, discloses for the first time the finding that the use of S-alkylisothiouronium derivatives before, during and/or after hemodialysis prevents vascular access thrombosis and/or extends the lifetime of vascular access points.

### Definitions

As used herein, the term “thrombosis” refers to formation of a blood clot inside a blood vessel, obstructing the flow of blood there through.

As used herein, the terms “vascular access point” are surgically created veins used to remove and return blood during hemodialysis and include the arteriovenous (AV) fistula and the AV graft.

As used herein the terms “fistula” and “arteriovenous fistula” refer to an artificial surgically created connection between an artery and a vein.

As used herein the terms “graft” and “arteriovenous graft” refer to a an artificial tubing, generally made out of teflon or fabric, that is attached on one end to an artery, and on the other end to a vein. The tube is placed entirely under the skin and the tube itself is punctured during dialysis.

As used herein, the term “vascular access thrombosis” refers to formation of a blood clots associated with arteriovenous fistula or grafts causing blockage of the access point and/or its dysfunction.

As used herein, the terms “subject” and “patient” may be interchangeably used and refer to a mammal, including both human and other mammals The methods of the present invention are preferably applied to human subjects. According to some embodiments, the subject may be suffering from thrombophilia. According to some embodiments, the subject may be a hemodialysis patient. According to some embodiments, the subject may suffer from hypertension.

As used herein, the term “thrombophilia” (also referred to as hypercoagulability or a prothrombotic state) is an abnormality of blood coagulation that increases the risk of blood clots in blood vessels.

As used herein, the term “hemodialysis” refers to a renal replacement therapy for lost kidney function, in subjects with kidney failure. The process involves removing waste and excess water from the blood.

As used herein, the term “hypotension” refers to a hemodynamic condition characterized by reduced blood pressure, which persists despite the maintenance of normal blood volume (normovolemia). Generally, a patient is suffering from hypotension when the mean arterial pressure is less than 90 mm Hg for at least one hour despite adequate ventricular filling pressures (pulmonary artery wedge pressure (PAWP) of at least 12 mm Hg) or despite a sufficient central venous pressure (CVP) of at least 8 mm Hg. Other indicators of hypotension are the failure of the hypotensive state to respond to aggressive initial fluid therapy (such as the administration of 500 ml of isotonic crystalloid, 25 gm or albumin, or 200 ml of other colloids (e.g. hydroxyethyl starch) or the need for pressor doses of dopamine (>5 g/kg/min), norepinephrine or other pressor agents to maintain a systolic blood pressure of 90 mm Hg.

The term “intradialytic hypotension (IDH)” is defined herein in patients with pre-dialysis blood pressure ≤120 mmHg as a decrease in systolic blood pressure (SBP) or mean arterial pressure (MAP) from the pre-dialytic baseline of both values. In some instances, the decrease is of about 20%.

As used herein, the term “drug induced hypertension” refers to hypertension caused by drugs administered to prevent and/or alleviate hypotension.

As used herein, the term “normotension” refers to a blood pressure that is within the normal range and or the normal patient blood pressure range.

As used herein, the term “predisposition for vascular access thrombosis” refers to patients who experience repeated clotting of their vascular access points and/or to patient's having more than two blocked, clotted and/or dysfunctional vascular access points.

As used herein the term “therapeutically effective amount” or “therapeutically efficient” as to a drug dosage, refer to dosage that provides the specific pharmacological response for which the drug is administered in a significant number of subjects in need of such treatment. The “therapeutically effective amount” may vary according, for example, the physical condition of the patient, the age of the patient and the severity of the condition.

The term “TXB104” as used herein refers to the injectable formulation of S-ethylisothiouronium diethylphosphate.

The term “about” as used herein refers to +/−10%.

As used herein, the term “alkylene” refers to a saturated or unsaturated hydrocarbon chain including straight chain or branched chain alkyl, alkenyl or alkynyl.

As used herein, the term “alkyl” refers to a saturated hydrocarbon chain containing 1 to 30, preferably 1 to 6 carbon atoms, such as, but not limited to, methyl, ethyl, n-propyl, isopropyl, n-butyl, isobutyl, tert-butyl, n-pentyl, n-hexyl, and the like. As used herein the term alkyl also reads on haloalkyls, which contain halogen atoms. Alkyl also includes heteroalkyl with heteroatoms of sulfur, oxygen and nitrogen.

“Alkenyl” and “alkynyl” are used to mean straight or branched chain hydrocarbon groups having from 2 to 12 carbons and unsaturated by a double or triple bond respectively, such as vinyl, allyl, propargyl, 1-methylvinyl, but-1-enyl, but-2-enyl, but-2-ynyl, 1 methylbut-2-enyl, pent-1-enyl, pent-3-enyl, 3-methylbut-1-ynyl, 1,1-dimethylallyl, hex-2-enyl and 1-methyl-1-ethylallyl.

The term “cycloalkyl” is used herein to mean cyclic radicals, including but not limited to, cyclopropyl, cyclopentyl, cyclohexyl, and the like.

The term “cycloalkylalkyl” as used herein refers to a cycloalkyl group appended to a lower alkyl radical, including, but not limited to cyclohexylmethyl. The “alkoxyalkyl” mentioned for R substitutes is preferably a group containing a total of 1-22 carbon atoms. As example, methoxyethyl, methoxypropyl, methoxybutyl, ethoxyethyl, ethoxypropyl, ethoxybutyl, n-propoxyethyl, and iso-propoxyethyl, can be mentioned.

The term “alkoxy” as used herein refers to an alkyl group attached to the parent molecular group through an oxygen atom.

The term “alkoxyalkoxy” as used herein refers to an alkoxy group attached to the parent molecular group through an alkoxy group.

The term “halo” or “halogen” as used herein refers to I, Br, Cl or F.

The term “carboxy” as used herein refers to the radical —COOH. The term “ester” refers to —COOR; and the term “amide” refers to —CONH₂ or —CONHR or —CONR₂. The term “cyano” as used herein refers to the radical —CN.

As used herein a “pharmaceutical composition” refers to a preparation of one or more of the compounds, described herein, or physiologically acceptable salts or prodrugs thereof, with other chemical components such as physiologically suitable carriers and excipients. The purpose of a pharmaceutical composition is to facilitate administration of a compound to an organism.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Without excluding other options, which are listed below, S-ethylisothiouronium diethylphosphate is at present the preferred compound for preventing vascular access thrombosis and/or for extending the lifetime of vascular access points, in hemodialysis patients. S-ethylisothiouronium diethylphosphate is now shown to be an effective agent for alleviating, preventing, inhibiting and/or reducing occurrence of thrombotic events, including vascular access thrombosis, in hemodialysis patients and/or for extending the lifetime of vascular access points.

According to another aspect of the present invention, there is provided a medicament for preventing a sequela of hypotension, the medicament comprising, as an active ingredient, a compound having the general formula (I):

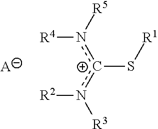

wherein,
R¹ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
and a pharmaceutically acceptable carrier or diluent.

According to some embodiments, the sequela of hypotension is selected from vascular access dysfunction, vascular access closure, vascular access thrombosis, drug induced hypertension and combinations thereof. Each possibility is a separate embodiment. According to some embodiments, vascular access dysfunction comprises arteriovenous fistula dysfunction. According to some embodiments, vascular access closure comprises arteriovenous fistula closure. According to some embodiments, vascular access thrombosis comprises arteriovenous fistula thrombosis. According to some embodiments, the subject is a hemodialysis patient.

According to another aspect of the present invention, there is provided an anti-thrombotic medicament for hemodialysis patients comprising, as an active ingredient, a compound having the general formula (I):

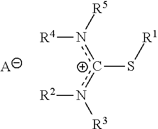

wherein,
R¹ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
and a pharmaceutically acceptable carrier or diluent.

According to some embodiments, the anti-thrombotic medicament is capable of preventing vascular access thrombosis. According to some embodiments, the anti-thrombotic medicament is capable of preventing arteriovenous (AV) fistula thrombosis

According to another aspect of the present invention, there is provided a medicament for extending the lifetime of vascular access points in hemodialysis patients, the medicament comprising, as an active ingredient, a compound having the general formula (I):

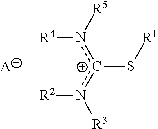

wherein,
R¹ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
and a pharmaceutically acceptable carrier or diluent.

According to some embodiments, the vascular access point is an AV fistula. According to some embodiments, extending the lifetime of a vascular access point comprises extending the lifetime of the access point by at least one week, by at least two weeks, by at least one month, by at least two months, or by at least six months, relative to an average lifetime of an untreated access point. Each possibility is a separate embodiment.

According to some embodiments, extending the lifetime of a vascular access point comprises extending the lifetime of the access point by at least one hemodialysis session, by at least two hemodialysis session, by at least three hemodialysis sessions, by at least five hemodialysis sessions, by at least 10 hemodialysis sessions, or by at least fifteen hemodialysis sessions relative to the average number of hemodialysis session per lifetime of an untreated vascular access point. Each possibility is a separate embodiment. As used herein, the term “hemodialysis session” refers to a single (typically out of three weekly) procedures of hemodialysis.

According to some embodiments, the physiologically acceptable anion is derived, without limitation, from a phosphorus containing acid, selected from the group consisting of acetate, adipate, alginate, citrate, aspartate, benzoate, benzenesulfonate, bitartarate, bisulfate, butyrate, camphorate, camphorsulfonate, digluconate, glycerophosphate, hemisulfate, heptanoate, hexanoate, fumarate, hydrochloride, 2-hydroxyethanesulfonate, isothionate, lactate, maleate, methanesulfonate, nicotinate, 2-naphthalenesulfonate, oxalate, palmoate, pectinate, 3-phenylpropionate, pivalate, propionate, succinate, tartrate, thiocyanate, phosphate, glutamate, bicarbonate, p-toluenesulfonate, chloride, bromide, iodide and undecanoate. Each possibility is a separate embodiment.

According some embodiments, the physiologically acceptable anion is an anion derived from a phosphorus containing acid. According to some embodiments, the anion is derived from a phosphorus acid ester or a phosphorus acid amide. According to some embodiments, the anion is derived from a mono or di-alkyl ester of a phosphorous containing acid.

Other examples of S-alkylisothiouronium derivatives, which can be used to alleviate, prevent, inhibit and/or reduce occurrence of thrombotic events in hemodialysis patients and/or to extend the lifetime of a vascular access point of hemodialysis patients, according to the present invention include, but are not limited to S-methylisothiouronium methylphosphite; S-methylisothiouronium dimethylphosphate; S-ethylisothiouronium metaphosphate; S-ethylisothiouronium ethylphosphite; S-ethylisothiouronium diethylphosphate; S-propylisothiouronium propylphosphite; S-isopropylisothiouronium metaphosphate; S-isopropylisothiouronium isopropylphosphite; S-butylisothiouronium dibutylphosphate; and S-isobutylisothiouronium isobutylphosphite. Each possibility is a separate embodiment.

These compounds are known to be safe for human use, as it is well known in the art that phosphorus containing derivatives of S-alkylisothiouronium have a low toxicity and their LD₅₀ (lethal dose 50%) is in the range of 100-1000 mg/kg, which is far above the therapeutic doses of these compounds.

The toxicological studies indicated that the compounds of the invention are not toxic when administered as either a single or repeated dose. For example, the LD₅₀ for TXB104 is up to 400 mg/kg in rats, values 300-400 fold higher than the therapeutically recommended dose of 0.1-2.4 mg/kg.

According some embodiments the medicament is administered before the hemodialysis procedure. According to other embodiments, the medicament is administered during the hemodialysis procedure. According to other embodiments, the medicament is administered after the hemodialysis procedure.

According to some embodiments, the therapeutically effective amount suitable for oral administration ranges between 0.1 and 2.4 mg/kg body weight.

According to another aspect of the present invention, there is provided a method for preventing a sequela of hypotension, the method comprising administering as an active ingredient, a compound having the general formula (I):

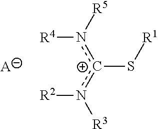

wherein,
R¹ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
and a pharmaceutically acceptable carrier or diluent.

According to some embodiments, the sequela of hypotension is selected from vascular access dysfunction, vascular access closure, vascular access thrombosis, drug induced hypertension and combinations thereof. Each possibility is a separate embodiment. According to some embodiments, vascular access dysfunction comprises arteriovenous fistula dysfunction. According to some embodiments, vascular access closure comprises arteriovenous fistula closure. According to some embodiments, vascular access thrombosis comprises arteriovenous fistula thrombosis. According to some embodiments, the subject is a hemodialysis patient.

In another aspect of the present invention there is provided a method for alleviating, preventing, inhibiting and/or reducing occurrence of thrombotic events, particularly vascular access thrombosis in hemodialysis patients. The method according to this aspect of the present invention is effected by administering to a subject a therapeutically effective amount of a compound having the general formula (I):

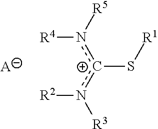

wherein
R¹ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
and a pharmaceutically acceptable carrier or diluent.

According to some embodiments, the thrombotic event comprises arteriovenous fistula thrombosis.

In another aspect of the present invention there is provided a method for extending the lifetime of a vascular access point. The method according to this aspect of the present invention is effected by administering to a subject a therapeutically effective amount of a compound having the general formula (I):

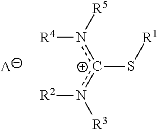

wherein
R¹ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
and a pharmaceutically acceptable carrier or diluent.

According to some embodiments, the vascular access point comprises an arteriovenous fistula. According to some embodiments, extending the lifetime of a vascular access point comprises extending the lifetime of the access point by at least one week, by at least two weeks, by at least one month, by at least two months, or by at least six months, relative to an average lifetime of an untreated access point. Each possibility is a separate embodiment.

According to some embodiments, extending the lifetime of a vascular access point comprises extending the lifetime of the access point by at least one hemodialysis session, by at least two hemodialysis session, by at least three hemodialysis sessions, by at least five hemodialysis sessions, by at least 10 hemodialysis sessions, or by at least fifteen hemodialysis sessions relative to the average number of hemodialysis session per lifetime of an untreated vascular access point. Each possibility is a separate embodiment. As used herein, the term “hemodialysis session” refers to a single (typically out of three weekly) procedures of hemodialysis.

### Pharmaceutical Composition of the Present Invention

A compound according to the present invention can be administered to a treated subject per se, or in a pharmaceutical composition where it is mixed with suitable carriers or excipients.

Pharmaceutical compositions may also include one or more additional active ingredients, such as, but not limited to, anti-hypotension agents and/or vasodilators.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes. Each possibility is a separate embodiment.

Pharmaceutical compositions for use in accordance with the present invention thus may be formulated in conventional manner using one or more physiologically acceptable carriers comprising excipients and auxiliaries, which facilitate processing of the active compounds into preparations, which, can be used pharmaceutically. Proper formulation is dependent upon the route of administration chosen.

For injection, the compounds of the invention may be formulated in aqueous solutions, carrier or diluent, preferably in physiologically compatible buffers, such as Hank's solution, Ringer's solution, phosphate buffer or physiological saline buffer. Each possibility is a separate embodiment.

For transmucosal administration, penetrants appropriate to the barrier to be permeated are used in the formulation. Such penetrants for example DMSO, or polyethylene glycol are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). Each possibility is a separate embodiment. If desired, disintegrating agents may be added, such as cross-linked polyvinyl pyrrolidone, agar, or alginic acid or a salt thereof, such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Each possibility is a separate embodiment. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. Each possibility is a separate embodiment. In addition, stabilizers may be added. All formulations for oral administration should be in dosages suitable for the chosen route of administration.

For buccal administration, the compositions may take the form of tablets or lozenges formulated in conventional manner.

For administration by inhalation, the compounds for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. Each possibility is a separate embodiment. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Pharmaceutical compositions for parenteral administration include aqueous solutions of the active preparation in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds, to allow for the preparation of highly concentrated solutions.

Alternatively, the active ingredient may be in powder form for constitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols. Each possibility is a separate embodiment.

Pharmaceutical compositions, suitable for use in context of the present invention, include compositions wherein the active ingredients are contained in an amount effective to achieve the intended purpose. More specifically, a therapeutically effective amount means an amount of a compound effective to prevent, alleviate, ameliorate, inhibit or reduce occurrence of thrombotic events, in particular vascular access thrombosis, in the subject being treated and/or an amount effective in treating hypotension while avoiding drug induced hypertension.

Determination of a therapeutically effective amount is well within the capability of those skilled in the art, especially in light of the detailed disclosure provided herein.

The exact formulation, route of administration and dosage can be chosen by the individual physician in view of the patient's condition. (See e.g., Fingl, et al., 1975, in “The Pharmacological Basis of Therapeutics”, Ch. 1 p. 1).

The amount of a composition to be administered will, of course, be dependent on the subject being treated, the severity of the thrombosis, the manner of administration, the judgment of the prescribing physician, etc. For example, doses up to 2.4 mg/kg of TXB104 are tolerated in healthy volunteers and represent a therapeutic alternative for the treatment of thrombosis, particularly vascular access thrombosis in hemodialysis patients.

A pharmaceutical composition containing S-alkylisothiouronium may be used either before, during or after the hemodialysis procedure. According to one embodiment the pharmaceutical composition of the invention is administered before the initiation of hemodialysis and it is especially preferred that the pharmaceutical composition of the invention is administered by intravenous injection or by oral administration before the hemodialysis procedure.

According to another embodiment of the invention, hemodialysis occurs with a dialyzer or dialysis tubing that is internally rinsed with a solution of S-alkylisothiouronium. According to a further embodiment of the invention, the administration of the amount of the S-alkylisothiouronium derivative is titrated to the blood pressure of the hemodialysis patient.

Single or multiple administrations of the compositions of the invention can be carried out. Furthermore, constant, variable, decreasing, or escalating doses may be employed.

Microparticles and nanoparticles can be used for sustained drug release in the present invention. Microparticles and nanoparticles employ small biodegradable spheres, which act as depots for delivery. The major advantage of polymer microspheres is that they are extremely safe and have been approved by the Food and Drug Administration in the US for use in human medicine as suitable sutures and for use as a biodegradable drug delivery system (Langer, 1990, Science, 249(4976):1527-33). The rates of polymer hydrolysis are very well characterized, which in turn allows for the manufacture of microparticles with sustained drug release over prolonged periods of time.

Administration of microparticles elicits long-lasting effect, especially if they incorporate prolonged release characteristics. The rate of release can be modulated by the mixture of polymers and their relative molecular weights, which will hydrolyze over varying periods of time.

Having now generally described the invention, the same will be more readily understood through reference to the following examples, which are provided by way of illustration and are not intended to be limiting of the present invention.

### EXAMPLES

### Example 1

### Formulations and Doses of Difetur

An example of an injectable formulation is presented in Table 1.

TABLE 1
Exemplary injectable formulation of the present invention (TXB104)

| Material | Quantity per ml | Function |
|---|---|---|
| S-ethylisothiouronium | 100 mg | Active ingredient |
| diethylphosphate | | |
| Monosodium Phosphate | 1.59 mg | Excipient, pH 5.0-6.0 |
| | | buffer component |
| Disodium Phosphate | 0.33 mg | Excipient, pH 5.0-6.0 |
| 7H₂O | | buffer component |
| Water for Injection | To make up | Excipient, Solvent |
| (WFI) | 1.00 ml | |

An example of an oral formulation is presented in Table 2.

TABLE 2
Exemplary oral formulation of the present invention

| Material | Quantity per tablet |
|---|---|
| S-ethylisothiouronium | 50 mg |
| diethylphosphate | |
| Lactose | 101 mg |
| Colloidal Silicone Dioxide | 1.0 mg |
| Microcrystalline cellulose | 40 mg |
| Crospovidone (PVP) | 4.0 mg |
| Stearic acid | 4.0 mg |
| Coating materials | Up to 5% of the weight of the |
| | compressed tablet core |

### Example 2

### TXB104 in Endstage Renal Disease (ESRD) Patients During Hemodialysis

I. Study Objectives

The purpose of the initial exploratory protocol was to analyze the efficacy of TXB104 in a first single dose 0.9 mg/kg administered as slow intravenous (IV) injection (10 ml diluted solution over 3 minutes). If no adequate blood pressure response was observed in first administration, a second dose of 1.2 mg/kg was administered, after a washout period of 72 hours. The study was conducted in ESRD patients with a predisposition for recurrent hypotensive episodes during hemodialysis sessions. The short-term safety and tolerability profile of TXB104 administered during hemodialysis was also evaluated and recorded in this set of patients. Plasma levels of TXB104 in ESRD patients on hemodialysis were measured, and the pharmacokinetic parameters were calculated.

Hemodynamic effects at baseline and during hemodialysis were recorded and monitored. Measured hemodynamic parameters were: systolic (SBP), diastolic (DBP), mean arterial blood pressure (MAP), Heart rate (HR), respiration rhythm, and oxygen saturation.

The number of intradialytic hypotensive episodes at baseline was recorded. The pre-dialysis and post-dialysis patient's weight, volume of fluids administered during dialysis, volume of fluids removed at end of dialysis, and change in scheduled length of dialysis session were recorded.

The changes in clinical manifestations commonly associated with intradialytic hypotension at baseline and during treatment with TXB104 were recorded. Common clinical manifestations associated with intradialytic hypotension included loss of consciousness, patient-reported nausea and vomiting, muscle cramps and sweating were recorded.

Primary safety parameters included: systolic and diastolic blood pressure, mean arterial blood pressure, heart rate and oxygen saturation, were measured at baseline, every 5 minutes for the first 30 minutes, thereafter every 10 minutes up to two hours, and every thirty minutes until the end of dialysis. After dialysis end these parameters were recorded at 1-hour intervals for 8 hours post dialysis. All hemodynamic readings were obtained directly from the monitor in triplicates. A printout of the hemodynamic parameters were printed, and used to analyze extreme values throughout the hemodialysis session.

II. Study Protocol

The study was performed as an open label study in hemodialysis patients with a history of several hypotension episodes during hemodialysis, using baseline characteristics of the same patients as control values. The patients received 0.9 mg/kg TXB104. The hemodialysis was started 10 min before the drug administration and was terminated 240 min after the drug administration. The stock solution of TXB104 was drawn using a 1 ml sterile disposable syringe, and was diluted with saline solution in a total volume of 10 ml. The total volume was injected slowly over 3 minutes to the port entering the body (and after the dialyzer).

III. Selection of Study Population

The records of all patients receiving maintenance hemodialysis as their means of renal replacement therapy at the Department of Hemodialysis at the Republican Hospital, Kishinev, Moldova was reviewed by the medical staff to identify all patients with a history of dialysis hypotension (>3 dialysis hypotensive event per month for the last six months prior to baseline). The medical records of all patients so identified were reviewed to determine eligibility according to the following inclusion/exclusion criteria.

Inclusion criteria: Patients aged 20-75 years inclusive were eligible for study participation if they experienced frequent bouts of hypotension (≥3 dialysis hypotensive events per month for the last six months prior to baseline) during dialysis despite standard adjustments and changes in anti-hypotensive medicine that would be instituted initially to treat the problem.

Exclusion criteria: Patients were excluded if they had uncontrolled hypertension >140/90 mm Hg, unstable angina, variable weight gains (an increase of more than 10 kg measured in between two consecutive dialysis), mental retardation, pregnancy, and malignancy or other concomitant serious diseases.

IV. The Effect of TXB104 on Blood Pressure During Hemodialysis

TXB104 (0.9 mg/kg) normalized blood pressure for approximately two hours during the hemodialysis session, requiring no additional medical intervention. For comparison, baseline (treatment without the drug) hemodynamic data were collected during two dialysis sessions in the same patients. These baseline data demonstrated that each of the hypotension predisposed patients required at least 3 to 4 medical interventions during the session to normalize the blood pressure. In contrast, in the presence of TXB104, the patients' blood pressure was significantly more stable during the hemodialysis. Unexpectedly and in contrast to other medical interventions the administration of TXB104 did not provoke drug induced hypertension and patients receiving TXB104 did not demonstrate any secondary hypertension incidents but retained normotensive values. In fact, normotension was maintained during the entire hemodialysis session, including immediately after TXB104 injection. This is in contrast to other sympathomimetic drugs, which result in an overshoot from hypotension to hypertension often causing secondary hypertension episodes, as schematically illustrated in FIG. 2.

### Example 3

### Reducing Vascular Access Thrombosis by Administering TXB104 to End Stage Renal Disease (ESRD) Patients

III. Study Objectives

The purpose of the initial exploratory protocol is to analyze the efficacy of TXB104 in reducing blood clotting in vascular access points of hemodialysis patient, by administering a single dose 0.9 mg/kg as slow intravenous (IV) injection (10 ml diluted solution over 3 minutes). The study is conducted on ESRD patients with recurrent vascular access thrombosis. Plasma levels of TXB104 in ESRD patients on hemodialysis are measured, and thrombotic occlusion, defined by absence of flow and inability to use the access for dialysis is evaluated. The evaluation for thrombotic occlusion is determined by the clinical team, which is unaware of the patient's thrombophilia status. Functioning vascular access is defined as successful cannulation with two needles and a minimum blood flow of 250 ml/min for at least one complete dialysis treatment. Patients with a history of failed access (i.e., fistula or graft created but never functioned) are eligible to participate as long as they have one permanent access that at some point functioned. Control subjects are patients who never had a thrombotic occlusion of a functioning permanent dialysis access.

Moreover, subclinical access dysfunction is retrospectively evaluated, as described by Miller et al. J Am Soc Nephrol 14: 2942-2947, 2003 and Ram et al. Kidney Int 64: 272-280, 2003. Patients with increased dialysis venous pressure, inability to achieve prescribed blood flow, swollen access site, prolonged bleeding from access needle site, or low urea reduction ratio were classified as having subclinical access dysfunction.

Pre-dialysis and post-dialysis patient's weight, volume of fluids administered during dialysis, volume of fluids removed at end of dialysis, and change in scheduled length of dialysis session are also recorded.

IV. Study Protocol

The study is performed as an open label study in hemodialysis patients, using baseline characteristics of the same patients as control values. The patients receive 0.9 mg/kg TXB104. The hemodialysis is started 10 min before the drug administration and is terminated 240 min after the drug administration. The stock solution of TXB104 is drawn using a 1 ml sterile disposable syringe, and is diluted with saline solution in a total volume of 10 ml. The total volume is injected slowly over 3 minutes to the port entering the body (and after the dialyzer).

IV. Selection of Study Population

The records of all patients receiving maintenance hemodialysis as their means of renal replacement therapy are reviewed by the medical staff to identify patients with recurrent vascular access dysfunction and/or vascular access thrombosis. The medical records of all patients so identified are reviewed to determine eligibility according to the following inclusion/exclusion criteria.
Inclusion criteria: Patients aged 20-75 years inclusive were eligible for study participation if they experience experiencing more than two vascular access thrombosis and/or dysfunction events per month for the last six months.
Exclusion criteria: Patients were excluded if they have uncontrolled hypertension >140/90 mm Hg, unstable angina, variable weight gains (an increase of more than 10 kg measured in between two consecutive dialysis), mental retardation, pregnancy, and malignancy or other concomitant serious diseases.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

1.-32. (canceled)

33. A method for preventing and/or reducing a sequela of intra-dialytic hypotension, comprising administering to a subject a therapeutically effective amount of a compound having the general formula I: 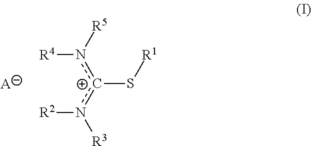
wherein,
R¹ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;
A⁻ is a physiologically acceptable anion;
and a pharmaceutically acceptable carrier or diluent;
wherein the sequela of hypotension is selected from the group consisting of vascular access dysfunction, vascular access closure, vascular access thrombosis, drug induced hypertension and any combination thereof.

34. The method of claim 33, wherein the vascular access comprises an arteriovenous fistula.

35. The method of claim 33, wherein preventing and/or reducing a sequela of intra-dialytic hypotension comprises extending the lifetime of a vascular access point.

36. The method of claim 35, wherein extending the lifetime of a vascular access comprises extending the lifetime of the access point by at least one week.

37. The method of claim 35, wherein extending the lifetime of a vascular access comprises extending the lifetime of the access point by at least three hemodialysis sessions.

38. The method of claim 33, wherein said physiologically acceptable anion is selected from the group consisting of an anion derived from a phosphorus containing acid, a phosphorous containing acid ester, a phosphorous containing acid amide, acetate, adipate, alginate, citrate, aspartate, benzoate, benzenesulfonate, bitartarate, bisulfate, butyrate, camphorate, camphorsulfonate, digluconate, glycerophosphate, hemisulfate, heptanoate, hexanoate, fumarate, 2-hydroxyethanesulfonate, isothionate, lactate, maleate, methanesulfonate, nicotinate, 2-naphthalenesulfonate, oxalate, palmoate, pectinate, 3-phenylpropionate, pivalate, propionate, succinate, tartrate, thiocyanate, phosphate, glutamate, bicarbonate, p-toluenesulfonate, chloride, bromide, iodide and undecanoate.

39. The method of claim 38, wherein the physiologically acceptable anion is a phosphorus containing acid.

40. The method of claim 39, wherein the phosphorus containing acid is selected from the group consisting of a mono-alkyl ester of a phosphorus containing acid and di-alkyl ester of a phosphorus containing acid.

41. The method of claim 33, wherein the compound is a S-alkylisothiouronium derivative having general formula (II): 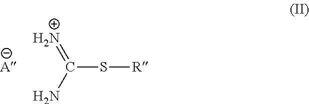
wherein
R″ is a straight or branched alkyl, optionally substituted by halogen; and
A″⁽⁻⁾ is an anion derived from a phosphorous containing acid.

42. The method of claim 33, wherein the compound is selected from the group consisting of:
S-methylisothiouronium methylphosphite; S-methylisothiouronium dimethylphosphate; S-ethylisothiouronium metaphosphate; S-ethylisothiouronium ethylphosphite; S-ethylisothiouronium diethylphosphate; S-propylisothiouronium propylphosphite; S-isopropylisothiouronium metaphosphate; S-isopropylisothiouronium isopropylphosphite; S-butylisothiouronium dibutylphosphate; and S-isobutyl-isothiouronium isobutylphosphite.

43. The method of claim 42, wherein the compound is S-ethylisothiouronium diethylphosphate.

44. The method of claim 33, wherein the compound is formulated for injection.

45. The method of claim 44, wherein said injectable therapeutically effective amount ranges between 0.1 and 2.4 mg/kg body weight.

46. The method of claim 45, wherein said injectable therapeutically effective amount ranges between 0.3 and 2.4 mg/kg body weight.

47. The method of claim 46, wherein said injectable therapeutically effective amount ranges between 0.5 and 1.8 mg/kg body weight.

48. The method of claim 46, wherein said injectable therapeutically effective amount ranges between 0.5 and 1.2 mg/kg body weight.

49. The method of claim 33, wherein the compound is formulated for oral administration.

50. The method of claim 49, wherein said oral therapeutically effective amount ranges between 0.1 and 2.4 mg/kg body weight.

51. The method of claim 33, wherein the compound is administered before the hemodialysis.

52. The method of claim 33, wherein the compound is administered during the hemodialysis.
